(19) United States
(12) Patent Application Publication
(10) Pub. No.: US 2013/0244316 A1
(43) Pub. Date: Sep. 19, 2013

(54) PAPER STRIP FOR DETERMINING MINIMUM INHIBITORY CONCENTRATIONS OF ANTIBIOTICS

(71) Applicant: Silvio BROCCO, Roseto Degli Abruzzi (TE) (IT)

(72) Inventor: Silvio BROCCO, Roseto Degli Abruzzi (TE) (IT)

(73) Assignee: LIOFILCHEM S.R.L., Roseto Degli Abruzzi (TE) (IT)

(21) Appl. No.: 13/886,825

(22) Filed: May 3, 2013

Related U.S. Application Data
(63) Continuation-in-part of application No. 13/392,308, filed on Feb. 24, 2012.

(30) Foreign Application Priority Data
Sep. 21, 2009 (IT) AN2009A000064

(51) Int. Cl. C12Q 1/18

(52) U.S. Cl. CPC C12Q 1/18
USPC 435/287.7

(57) ABSTRACT

An article for determining a minimum inhibitory concentration of antibiotics. The article has a strip of paper in which the strip of paper is permeable to air so as to prevent air bubbles from forming at a point of contact with a microbial culture medium. The strip of paper has a predetermined concentration gradient of an antibacterial agent graded on a scale of fifteen dilution units. The antibacterial agent on the strip of paper is releasable slowly from the strip of paper. The scale of fifteen dilution units can he expressed on a color scale and can be expressed in μg/mL. The strip of paper is impregnated with the antibacterial agent.

### CROSS-REFERENCE TO RELATED U.S. APPLICATIONS

The present application is a continuation-in-part of U.S. application Ser. N. 13/392,308, filed on Feb. 24, 2012, and entitled “Paper Strip for Determining Minimum Inhibitory Concentrations of Antibiotics”, presently pending.

### STATEMENT REGARDING FEDERALLY SPONSORED RESEARCH OR DEVELOPMENT

Not applicable.

### NAMES OF PARTIES TO A RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to chemical, clinical, bacteriological and immunological analyses. More particularly, the present invention the relates to impregnated paper that is used to determine, in an economical and accurate manner, the correct minimum inhibitory concentration of antibiotic molecules to be administered to patients.

2. Description of Related Art Including Information Disclosed Under 37 CFR 1.97 and 37 CFR 1.98.

One of the main objectives of medicine is the determine the correct dosage of antibiotics required both to obtain the greatest effect in order to eradicate the infections and to prevent large doses of antibiotics from having adverse reactions on the patient.

Initially, this problem was resolved in medicines by means of the CLSI (Clinical and Laboratory Standard Institute) standard microdilution method for dosing the antibiotic with progressively decreasing concentrations. The problem with this method is that it takes a very long time to determine the minimum inhibitory concentration. This is the result of the need to carry out numerous manual laboratory tests in order to identify the correct dose of antibiotic to be administered to the patient.

European Patent Application No. EP 0157 071 describes a method of determining of the minimum inhibitory concentration to be administered to the patient. This European Patent describes the use of a graded carrier with a scale of concentrations of one or more clinically or biologically active substances. As a result, the substance on this carrier is transferred onto the surface of a culture medium containing bacteria taken from the patient. As such, it is possible to determine the minimum inhibitory concentration. A specific concentration pattern is obtained by applying a number of units, which contain the substance or substances in known concentrations, to the surface of a medium in such a way that the specific concentration pattern is obtained in a rectangular area. This European Patent utilizes a non-permeable porous or non-porous material, for example, cellulose, polyacrylamide, polyester, polyamide or material. These materials can be opaque or transparent. This European Patent does not disclose a paper strip for determining the minimum inhibitory concentration of antibiotics which is impregnated with a predetermined concentration of an antibacterial agent grated On a scale of fifteen dilution intervals. This European Patent also teaches that the strip is impermeable to air rather than permeable to air.

A company OXOID Ltd. Provides a product known as the M.I.C. Evaluator. This product is based upon a similar principle as European Patent Application No. EP 0157071. In particular, this product utilizes a polymeric strip graded with a concentration scale of numerous clinically or biologically active agents.

Both European Patent Nos. EP 0157071 and the M.I.C. Evaluator provide carriers that are made of plastic and are difficult to remove from the wrapping Which contains them due to the fact that they are very thin. This makes them more sensitive to the electrostatic attraction forces typical of thin plastic layers. As a result, specific and expensive equipment is required. Another problem is that the plastic material that is used for the carrier is impermeable to air. As a result, incorrect applications to the microbial culture medium can create air bubbles which may not be visible to the naked eye but which could invalidate the test, or render it inadequate. Additionally, the use of such a plastic carrier causes it to immediately start to release antibiotics when it comes into contact with agar surface of the microbial culture medium. As a result, this makes its correct repositioning on the microbial culture medium impossible.

U.S. Pat. No. 4,778,758 describes a device for the determination of microorganisms. This patent describes a sealed rectangular container that contains a transparent non-porous strip for the determination of the test.

It is an object of the present invention to provide a paper strip for determining the minimum inhibitory concentration of antibiotics that is easy to remove from the wrapping which contains the test strip.

It is another object of the present invention to provide an article for determining a minimum inhibitory concentration of antibiotics which insensitive to electrostatic attraction forces.

It is another object of the present invention to provide an article for determining a minimum inhibitory concentration of antibiotics which is permeable to air.

It is a further object of the present invention to provide an article for determining a minimum inhibitory concentration of antibiotics which avoids invalid test results.

It is still another object of the present invention to provide an article for determining a minimum inhibitory concentration of antibiotics which serves to release the antibiotics very slowly.

It is still a further object of the present invention to provide an article for determining a minimum inhibitory concentration of antibiotics which avoids the use of specific and expensive equipment.

It is still another object of the present invention to provide an article for determining a minimum inhibitory concentration of antibiotics which avoids incorrect applications to the microbial culture medium and avoids the creation of air bubbles which could invalidate the test or make the test inaccurate.

It is still a further object of the present invention to provide an article for determining a minimum inhibitory concentration of antibiotics which does not immediately release the antibiotics upon coming into contact with the agar surface of the microbial culture medium.

These and other objects and advantages of the present invention will become apparent from a reading of the attached specification and appended claims.

### BRIEF SUMMARY OF THE INVENTION

The present invention is an article for determining a minimum inhibitory concentration of antibiotics. The article of the present invention includes a strip of paper in which the strip of paper is permeable to air so as to prevent air bubbles from forming, at a point of contact with a microbial culture medium. This strip of paper has a predetermined concentration gradient of an antibacterial agent graded on a scale of fifteen dilution intervals.

The antibacterial agent on the strip of paper is releasable slowly from the strip of paper. A silica gel container receives the strip of paper therein so as to protect the strip of paper from moisture. The scale of fifteen dilution intervals is on a color scale. This color scale can be expressed in micrograms, i.e. μg/mL. The strip of paper is impregnated with the antibacterial agent.

Since the present invention utilizes a paper strip that is printed with a known numerical sequence (corresponding to a different grading of the impregnated antibiotic), the present invention is able to simplify its extraction from the blister packing or from the wrapping which normally protects it. The present invention also avoids the creation of air bubbles between the microbial cultural medium and the paper strip itself. As such, the present invention avoids the possibility of invalidating the test or rendering the test inaccurate.

Since the paper strip of the present invention is impregnated with antibiotics, the paper strip has the characteristic of gradually releasing the antibiotics once positioned in the microbial culture medium. This facilitates its repositioning on the microbial culture medium. As such, the present invention reduces the costs associated with determining the minimum inhibitory concentration.

The present invention utilizes he paper strip in order to simplify the execution of the relative test due to the fact that the strip of paper is thicker than strips of plastic and because the strip of paper is permeable to air. The strip of paper posses the characteristic of releasing the antibiotics very slowly once positioned on the microbial culture medium. This facilitates its repositioning, if required, on the microbial culture medium.

This foregoing Section is intended to describe, in generality, the preferred embodiment of the present invention. It is understood that modifications to this preferred embodiment can be made within the scope of the present invention. As such, this Section should not to be construed, in any way, as being limiting of the broad scope of the present invention. The present invention should only be limited by the following claims and their legal equivalents.

### BRIEF DESCRIPTION OF THE DRAWINGS

FIG. 1 shows the article of the present invention impregnated with the predetermined concentration of the antibacterial agent.

FIG. 2 shows the configuration of the zone created by placing the article of the present invention on a microbial culture medium.

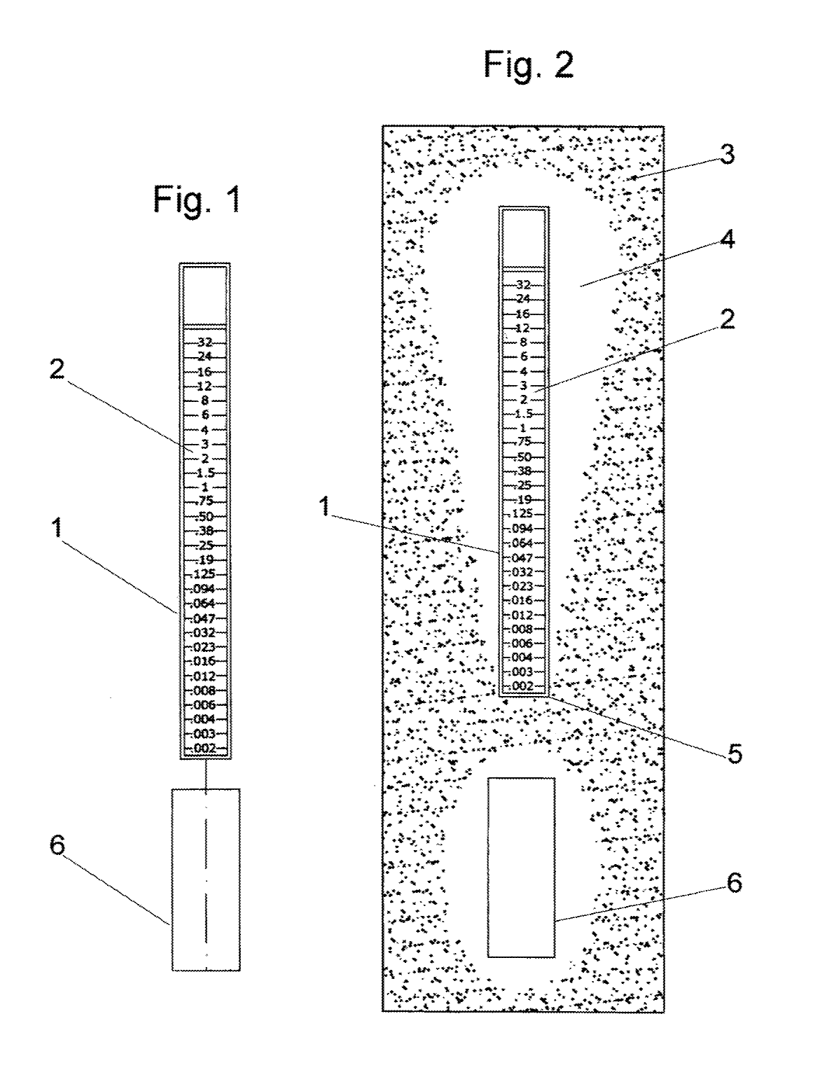

### DETAILED DESCRIPTION OF THE INVENTION

The present invention provides a strip of paper 1 impregnated with a predetermined concentration gradient of an antibacterial agent graded on a color scale of fifteen dilution intervals. These dilution intervals are expressed in μg/mL 2 so as to determine the minimum inhibitor concentration at a zone 4 which intersects the strip 1. The strip 1 is positioned on a microbial culture medium of bacteria 3 that is taken from the patient. A point 5 is at a given value reading of the antibacterial agent and facilitates its extraction from the relative blister packing or wrapping because of its greater thickness compared to that of plastic strips currently in use. The strip 1 also avoids the reciprocal force of electrostatic attraction.

The paper strip 1 has the characteristic of being permeable to air. This prevents air bubbles from forming at the point of contact between the microbial culture medium 3 and the paper strip 1. As such, invalid tests or inaccurate test results are avoided.

The paper strip 1 begins to release the antibiotics very slowly when it first conies into contact with the microbial culture medium 3. This allows the user to reposition the paper strip 1 easily on the microbial culture medium 3 so as to determine of the minimum inhibitory concentration.

The paper strip 1 is equipped with a silica gel container 6. This serves to preserve the paper strip 1 from contact with moisture and avoids the contamination of the predetermined concentrations of the antibacterial agent from moisture. The predetermined concentrations of the antibacterial agent are shown on a color scale of fifteen dilution intervals expressed in μg/mL 2.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. An article for determining a minimum inhibitory concentration of antibiotics, the article comprising:
a strip of paper in which the strip of paper is permeable to air so as to prevent air bubbles from forming at a point of contact with a microbial culture medium, said strip of paper having a predetermined concentration gradient of an antibacterial agent graded on a scale of fifteen dilution intervals.

2. The article of claim 1, the antibacterial agent on said strip of paper being releasable slowly from said strip of paper.

3. The article of claim 1 further comprising:
a silica gel container receiving said strip of paper therein so as to protect said strip of paper from moisture.

4. The article of claim 1, said scale of fifteen dilution intervals being on a color scale.

5. The article of claim 4, said color scale expressed in μg/mL.

6. The article of claim 1, said scale of fifteen dilution intervals being expressed in μg/mL.

7. The article of claim 1, said strip of paper being impregnated with said antibacterial agent.
